(19) United States
(12) Patent Application Publication
(10) Pub. No.: US 2022/0221434 A1
(43) Pub. Date: Jul. 14, 2022

(54) ANALYTICAL DEVICE AND ANALYTICAL METHOD

(71) Applicants: OSAKA UNIVERSITY, Suita-shi (JP); SHIMADZU CORPORATION, Kyoto-shi (JP)

(72) Inventors: Fumio MATSUDA, Suita-shi (JP); Shinji KANAZAWA, Kyoto (JP)

(21) Appl. No.: 17/609,622

(22) PCT Filed: May 7, 2020
(86) PCT No.: PCT/JP2020/018586

§ 371 Date: Nov. 8, 2021

(30) Foreign Application Priority Data
May 10, 2019 (JP) 2019-089616

(51) Int. Cl. G01N 30/86; G06F 16/33

(52) U.S. Cl. CPC G01N 30/8675; G01N 30/72; G06F 16/334

(57) ABSTRACT

The present invention is an analytical device (50) including: an information acquisition unit (51) configured to acquire first identification information identifying, from a result of measuring an analyte contained in a biological sample using an analyzer (10), the analyte; an extraction unit (56) configured to extract a related term related to the analyte, from a database (41) in which document data is accumulated, on a basis of the first identification information acquired by the information acquisition unit; and a presentation unit (57, 59) configured to present the related term extracted by the extraction unit to a user.

### TECHNICAL FIELD

The present invention relates to an analytical device and an analytical method.

### BACKGROUND ART

In the biomedical field, in order to identify various metabolites contained in a biological sample, multi-component simultaneous analysis using a gas chromatograph mass spectrometer (GC/MS) or a liquid chromatograph mass spectrometer (LC/MS) is performed.

Analysis data output from an analyzer such as GC/MS or LC/MS is comprehended by a user to identify a metabolite. The comprehension of the analysis data largely depends on the knowledge and experience of the user. Therefore, there has been proposed a metabolite analytical data processing device that makes work related to identification of various metabolites contained in a biological sample efficient and makes verification of analysis data efficient (see Patent Literature 1). In the device of Patent Literature 1, a chart called a metabolic map describing metabolic pathways, which is used in the field of handling metabolism in vivo, is used. In the metabolic map, chemical reactions occurring in the process of metabolism, various compounds (metabolites) generated by the reactions, enzymes involved in the reactions, and the like are listed, and the flow of metabolism can be understood at a glance. In the device of Patent Literature 1, information regarding a metabolite contained in a biological sample, an analysis condition capable of analyzing a certain metabolite, and the like is clearly indicated on a metabolic map, thereby supporting the user to set an analysis condition and grasp information of the metabolite.

In order to analyze the identification result of metabolites based on the analysis data of the analyzer and then obtain valuable information, a tool corresponding to the analysis target is further used. Therefore, software as an analytical tool such as lipidomics (lipid molecule (lipidome) analysis), proteomics (protein analysis), and metabolomics (metabolome analysis) is individually developed by researchers and companies. Furthermore, in recent years, an information platform conforming to an application programming interface (API) or the like has been provided in order to comprehensively analyze various types of omics data and to make data compatible among various types of software used in the biomedical field (see Non Patent Literature 1).

In addition, research results on various discoveries in the biomedical field have been disclosed in various public databases. One such database is MEDLINE operated by the National Library of Medicine (NLM) which stores medical literature information. Literature information stored in MEDLINE can be searched using, for example, a search function of PubMed provided by the National Library of Medicine on the Web (see Non Patent Literature 2). In order for the search to be appropriately performed, the bibliographical information of every literature stored in MEDLINE is indexed by MeSH (Medical Subject Heading) which is a medical literature thesaurus. The indexing is performed by assigning a MeSH term and a MeSHID to the bibliographical information of each literature. A plurality of MeSH terms may be assigned to one literature. A MeSHID is associated with a MeSH term depending on the category of the MeSH term.

In the biomedical field, the relevance between a gene, a drug, a disease, and the like may be analyzed from analysis data of a biological sample to estimate a gene expression control mechanism and an intermolecular interaction. In order to estimate the gene expression control mechanism and the intermolecular interaction, it is required to construct a map or a model by reading scientific literatures related to biopharmaceuticals. The quantity of scientific literatures is enormous, and scientific literatures are described in different perspectives in many subdivided specialized areas. Such work of linking the contents described in the scientific literatures to each other is difficult for a person to perform. Therefore, a method has been proposed in which terms related to genes, drugs, diseases, and the like described in various scientific literatures are extracted from PubMed using MeSH terms, and their relevance is tabulated to estimate a gene expression control mechanism or an intermolecular interaction (see Non Patent Literature 3).

### CITATION LIST

### Patent Literature

Patent Literature 1: JP 2010-216981 A

### Non Patent Literature

Non Patent Literature 1: Garuda Platform, Specified non-profit organization, System Biology Research Organization, [online], [searched on Apr. 21, 2019], Internet <http://www.garuda-alliance.org/about.html>
Non Patent Literature 2: PubMed, [online], [searched on Apr. 17, 2019], Internet <URL: https://www.ncbi.nlm.nih.gov/pubmed>
Non Patent Literature 3: Stephen Joseph Wilson et al., ‘Automated literature mining and hypothesis generation through a network of Medical Subject Headings’, [online], bioRxiv, [searched on Apr. 17, 2019], Internet, <URL: https://www.biorxiv.org/content/10.1101/403667v1>

### SUMMARY OF INVENTION

### Technical Problem

Statistical theories and computer science technologies are used to develop and improve analytical software for data analysis in the biomedical field. A researcher who is familiar with statistics and computer science can derive some results using some analytical method on the basis of given analysis data. However, such results derived from the analysis data may not be biologically meaningful. That is, unless one is familiar with the meaning of the analysis data and its background, it is not possible to determine whether such an analytical method is appropriate, and to obtain a meaningful analytical result for a researcher in the biomedical field.

In multiomics in which changes in genes, proteins, metabolites, and the like are integrally analyzed, the number of literatures that researchers should refer to in order to analyze their respective omics data is huge, and the number is increasing every day. Even if the relevance of gene-gene, disease-gene, and drug-gene is obtained as knowledge by the method (data mining) described in Non Patent Literature 3, it is necessary for researchers to read relevant literatures and judge by themselves in order to utilize the knowledge. However, it is difficult for the researchers to efficiently extract meaningful literatures from the huge amount of literatures.

Here, the problem in the case of analyzing the analysis data of the biological sample has been described, but there is a similar problem in the case of extracting a literature meaningful for investigating the cause of environmental pollution from the measurement result of substances such as environmental hormones contained in a sample other than the biological sample, for example, a liquid sample collected from sea water, lake water, river, or the like.

The present invention has been made to solve the above problems, and an object is to facilitate extracting document data meaningful for understanding measurement result of substances contained in a sample using an analyzer.

### Solution to Problem

A first aspect of the present invention is an analytical device including: an information acquisition unit configured to acquire first identification information for identifying, from a result of measuring an analyte contained in a sample using an analyzer, the analyte; an extraction unit configured to extract a related term related to the analyte, from a database in which document data is accumulated, on a basis of the first identification information acquired by the information acquisition unit; and a presentation unit configured to present the related term acquired by the extraction unit to a user.

A second aspect of the present invention is an analytical method including: a step of acquiring a result of measuring an analyte contained in a sample using an analyzer; a step of acquiring first identification information for identifying the analyte from the result of measuring the analyte; a step of extracting a related term related to the analyte from a database in which document data is accumulated on a basis of the first identification information; and a presentation step of presenting the related term to a user.

### Advantageous Effects of Invention

According to the present invention, a related term is extracted from the database in which the document data is accumulated using the first identification information acquired from the measurement result of the analyte contained in the sample, and the extracted related term is presented to the user. Therefore, the user can easily search the database for the document data meaningful for understanding the measurement result of the analyte using the presented related term.

### BRIEF DESCRIPTION OF DRAWINGS

FIG. 1 is a schematic diagram of an information providing system including an analytical device 50 according to an embodiment of the present invention.

FIG. 2 is an explanatory diagram illustrating an example of processing of creating analytical data from analysis data.

FIG. 3 is a view showing a display example of a metabolic map.

FIG. 4 is a block diagram illustrating an example of a schematic configuration of an analytical device 50.

FIG. 5 is a flowchart illustrating an example of processing of the analytical device 50.

FIG. 6 is a diagram illustrating an example of a MeSHID group which is a set of MeSHID which is identification information of an analyte.

FIG. 7 is a diagram illustrating a display example of a result of association analysis.

FIG. 8 is a flowchart illustrating another example of processing of the analytical device 50.

FIG. 9 is a diagram showing an example of a first MeSHID group and a second MeSHID group.

FIG. 10 is a diagram illustrating a display example of a result of association analysis.

### DESCRIPTION OF EMBODIMENTS

### [Outline of System Including Analytical Device]

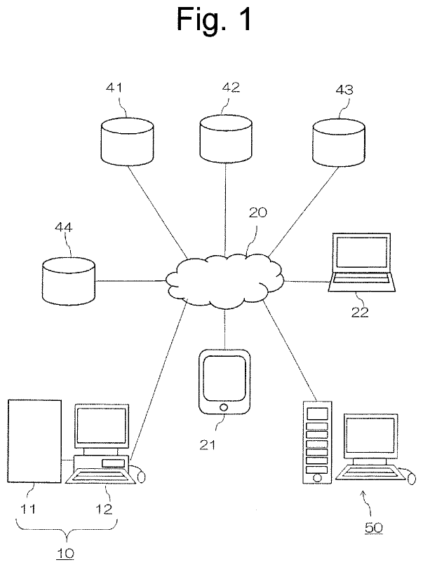

Hereinafter, embodiments of the present invention will be described with reference to the drawings. FIG. 1 is a schematic diagram of an information providing system including an analytical device 50 according to this embodiment.

The information providing system includes a plurality of terminal devices in which at least one piece of software for realizing processing desired by the user is installed, and a plurality of databases 41, 42, 43, and 44 for providing information in response to an inquiry from the terminal device. Document data is accumulated in each of the plurality of databases 41, 42, 43, and 44. The terminal device is a computer device such as a tablet terminal 21 or a personal computer 22.

The analyzer 10 includes a device main body 11 that executes analysis by a mechanical operation, and a personal computer 12 in which control software for controlling the operation of the device main body 11, processing software for processing data obtained by the device main body 11 executing analysis, and the like are installed. The terminal devices 21 and 22, the personal computer 12, and the databases 41, 42, 43, and 44 are connected via the Internet 20. Analysis data of the analyzer 10 is stored in a storage device of the personal computer 12 of the analyzer 10. The personal computer 12 can transmit and receive data to and from the terminal devices 21 and 22 and the analytical device 50 via the Internet 20. An entity of the analytical device 50 is a computer device such as a personal computer or a workstation. The analytical device 50 performs analysis for allowing a user to search at least one database on the basis of analysis data of a sample acquired by the analyzer 10, and providing the user with a term that helps the user to obtain document data necessary for comprehending the analysis data.

Various databases corresponding to types of samples that can be analyzed by the analyzer 10 are used as databases available via the Internet 20. Examples of a database used in the case of a biological sample include a gene database, a protein information database, a pharmaceutical information database, a medical literature database, and the like. Examples of the medical literature database include a medical literature database MEDLINE operated by the National Library of Medicine (NLM). The document data accumulated in the database includes papers, books, dictionaries, pharmaceutical package inserts, and the like.

As the analyzer 10, a chromatograph such as liquid chromatography (LC) or gas chromatography (GC), and a chromatograph mass spectrometer such as LC/MS or GC/MS in which a mass spectrometer is combined with a chromatograph can be used. When the analyzer 10 is a chromatograph mass spectrometer, graphs such as a chromatogram and a mass spectrum are acquired as analysis data. Coordinate data (for example, numerical data that is a set of a retention time and a signal intensity, a set of a mass-to-charge ratio m/z value and a signal intensity, or the like) representing each point on the graph may be acquired as analysis data. In short, any form of analysis data may be used as long as the type and amount of the analyte contained in the sample can be specified based on the analysis data. Examples of the sample to be provided to the analyzer 10 include a liquid sample and a gas sample. Examples of the liquid sample include urine and blood of animals including humans, and biological samples such as crude extracts obtained by breaking down cellular structures of organisms. When the sample is a biological sample, the analyte is a metabolite, a protein, a compound, or the like.

### [Configuration of Analytical Device]

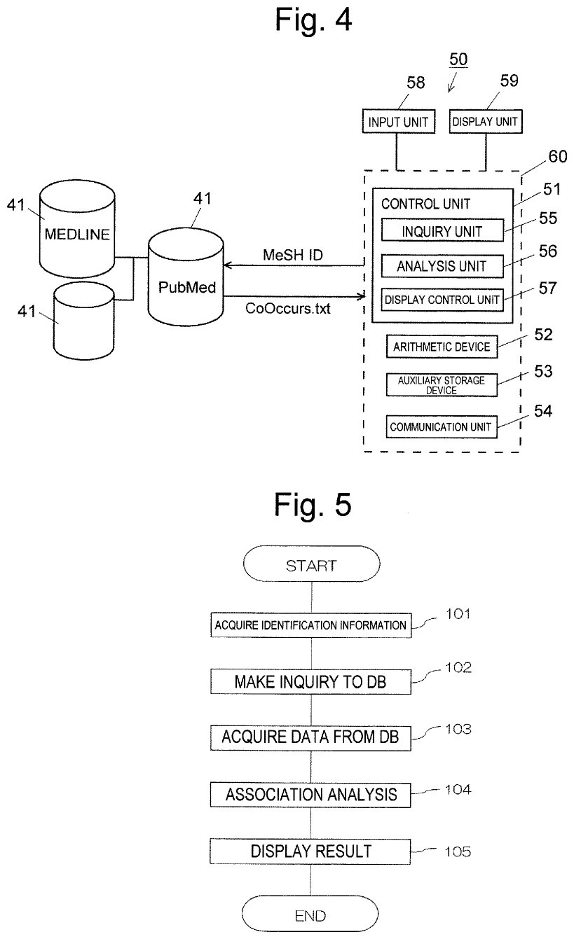

FIG. 4 is a block diagram illustrating a schematic configuration of the analytical device 50.

The analytical device 50 includes a device main body 60, and an input unit 58 and a display unit 59 connected to the device main body 60. The device main body 60 includes a control unit 51, an arithmetic device 52 such as a CPU that executes various arithmetic processing, an auxiliary storage device 53 that stores an analysis result and the like, and a communication unit 54 that transmits and receives data to and from a database 41 via the Internet 20. In FIG. 4, MEDLINE, and PubMed, which is a search engine of literatures recorded in MEDLINE, are illustrated as the database 41. The control unit 51 includes an inquiry unit 55, an analysis unit 56, and a display control unit 57 as functional blocks. Furthermore, the control unit 51 controls the operations of the arithmetic device 52, the auxiliary storage device 53, and the communication unit 54.

An entity of the analytical device 50 is a personal computer, and each function of the control unit 51 is embodied when dedicated software installed in advance in the personal computer is executed on the computer. The input unit 58 is a keyboard or a pointing device (mouse or the like) attached to the computer. The display unit 59 is a display monitor of the computer. The auxiliary storage device 53 is a hard disk drive (HDD), a solid state drive (SSD), or the like. The memory 51, the arithmetic device 52, the auxiliary storage device 53, the communication unit 54, and the control unit 51 are connected by an internal bus.

### [Analysis Processing by Analytical Device]

Next, analysis processing executed by the analytical device 50 will be described.

### [Creation of Analytical Data]

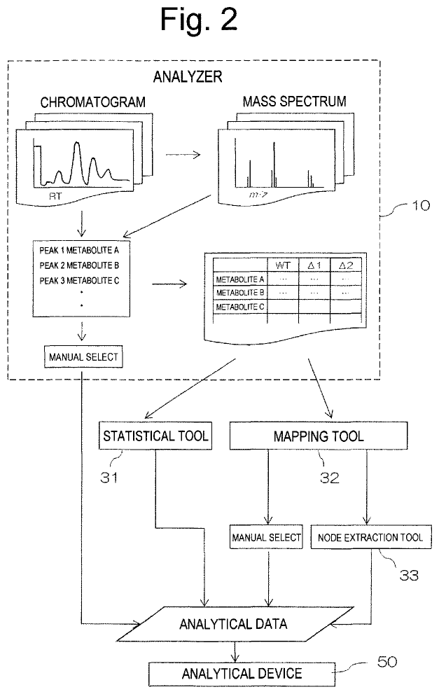

Information for identifying a substance (analyte) to be analyzed by the analytical device 50 among substances contained in the sample provided to the analyzer 10 is input to the analytical device 50 as analytical data. Therefore, in a case where the analysis data is analytical data as it is depending on the format of the analysis data acquired by the analyzer 10, processing of processing the analysis data or processing of extracting the analyte from the analysis data to create the analytical data may be required. FIG. 2 is an explanatory diagram illustrating an example of processing of creating analytical data from analysis data of the analyzer 10. In the present embodiment, the processing of creating analytical data is performed by a terminal device in which predetermined software necessary for the processing is installed. Therefore, prior to the processing of creating the analytical data, the user transmits the analysis data from the analyzer 10 to the terminal device.

Here, the processing of creating analytical data from data obtained by analyzing a cell extract of Saccharomyces cerevisiae by LC/MS will be described as an example. The cell extract of Saccharomyces cerevisiae is a crude extract obtained by culturing a wild strain (WT), a mutant strain (Δ1) obtained by knocking out a specific gene involved in the metabolism of Saccharomyces cerevisiae, and a mutant strain (Δ2) obtained by knocking out a specific gene involved in the metabolism of Saccharomyces cerevisiae, which is different from the mutant strain (Δ1), under the same conditions, and then destroying the cells. The analysis data is data obtained by analyzing the cell extracts of each strain of Saccharomyces cerevisiae by LC/MS under the same analysis conditions in order to compare the metabolites of the strains. Typically, the data is a chromatogram or a mass spectrum, but may be numerical data including a set of a retention time and a signal intensity or numerical data including a set of an m/z value and a signal intensity.

In LC, the retention time (RT) of the component in the sample is determined from the properties of the column and the elution conditions. If the retention time of a substance known as a metabolite of Saccharomyces cerevisiae is known, the metabolite contained in each cell extract can be identified from the retention time of the peak position of the chromatogram obtained for the cell extract of each strain of Saccharomyces cerevisiae. Even if the metabolite cannot be identified from the retention time of the chromatogram, the metabolite contained in each cell extract can be identified by comparing the theoretical m/z value of the known metabolite calculated in advance with the m/z value of the peak of the mass spectrum. In addition, the amount of metabolites contained in each cell extract can be calculated from the area (height) of each peak of the chromatogram. Therefore, by comparing the chromatograms and mass spectra obtained for each of the wild strain (WT), the mutant strain (Δ1), and the mutant strain (Δ2), metabolites satisfying specific conditions can be selected, such as selecting metabolites with different amounts contained in the cell extract between the wild strain (WT) and the mutant strain (Δ1) or the mutant strain (Δ2), or selecting metabolites commonly contained in a large amount in the three strains. The analytical data includes names of one or more selected metabolites.

The task of selecting a metabolite that satisfies a specific condition may be performed, for example, by a user manually selecting a peak on a chromatogram. In addition, a metabolite satisfying a specific condition may be automatically or manually selected from a result of analyzing analysis data using a predetermined analytical tool.

When the chromatograms of the wild strain (WT) and the mutant strain (Δ1) (or the mutant strain (Δ2)) can be visually recognized to determine that the peak areas are clearly different between the wild strain and the mutant strain, the user can manually select the peak. When a peak is selected, the terminal device identifies a metabolite corresponding to the peak.

Examples of the analytical tool include a statistical tool 31 and a mapping tool 32. The statistical tool 31 is a tool that analyzes a correlation between a plurality of variables on the basis of data regarding the variables using a statistical method such as multivariate analysis. Using the statistical tool 31, for example, metabolites having a significant difference between the mutant strains (Δ1) (or mutant strains (Δ2)) as compared to the wild strain (WT) can be automatically selected.

The mapping tool 32 is a tool that creates a metabolic map in which metabolic pathways are schematized. Using the mapping tool 32, for example, it is possible to create a metabolic map incorporating quantitative values of metabolites contained in cell extracts of a wild strain (WT), a mutant strain (Δ1), and a mutant strain (Δ2), and to visualize quantitative changes of metabolites generated by knocking out a specific gene.

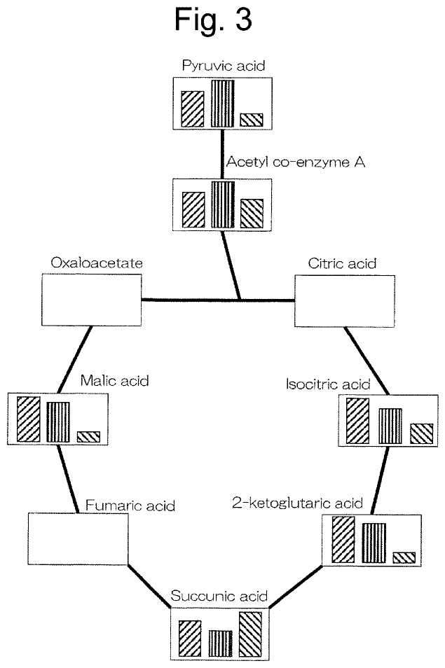

FIG. 3 is a diagram illustrating a display example of a metabolic map. In this display example, the names of metabolites generated in each reaction of the tricarboxylic acid cycle and the bar graphs representing the quantitative values of the wild strain (WT), the mutant strain (Δ1), and the mutant strain (Δ2) of each metabolite are shown. The bar graph shows quantitative values of the wild strain (WT), the mutant strain (Δ1), and the mutant strain (Δ2) arranged in this order from the left on the drawing. Due to the nature of the LC column, the graph column is blank for undetectable metabolites. In this display example, the quantitative change of each metabolite caused by the difference between the wild strain and the mutant strain is represented by a graph. Therefore, while viewing the graph on the TCA cycle, the user can manually select metabolites whose amount is clearly reduced in the mutant strain (Δ2) than in the wild strain (WT), for example.

In the metabolic map of FIG. 3, only the names of metabolites of the TCA cycle are displayed, but names of enzymes that catalyze reactions between metabolites, genes related to reactions, proteins, and the like may be simultaneously displayed. In addition, the relevance of metabolites, catalysts related to metabolism, genes, proteins, and the like on the metabolic map may be represented by nodes and edges, and the nodes may be extracted using a node extraction tool 33, whereby metabolites, catalysts, genes, proteins, and the like that satisfy specific conditions can also be selected. In this case, in addition to the name of the metabolite or instead of the name of the metabolite, the names of the catalyst, the gene, the protein, and the like are included in the analytical data.

Examples of the mapping tool 32 include a tool also called a network visualization tool that extracts and visualizes knowledge networks, in addition to a tool that outputs a metabolic map as illustrated in FIG. 3. In the network visualization tool, information such as quantitative increase/decrease of metabolites obtained by statistically processing analysis data is combined with a network in which correlations of metabolites, catalysts, genes, proteins, and the like are indicated by nodes and edges, whereby it is possible to visualize which node and edge occupy a relatively important position in the network and where the node and edge are located. Also for such a network, by extracting nodes using the node extraction tool 33, metabolites, catalysts, genes, proteins, and the like that satisfy specific conditions can be selected.

In the terminal device, when one or a plurality of analytes such as metabolites, catalysts, genes, and proteins are selected by the above-described method, their names are set as analytical data. The set analytical data is sent from the terminal device to the analytical device 50 via the Internet 20. In addition, the analytical data may include an ID assigned in advance for identification of the analyte together with the name of the analyte.

For example, in a metabolite database referred to for identifying metabolites or the like from a graph (chromatogram, mass spectrum, and the like) obtained by the analyzer 10, an ID (metabolite ID) for identifying metabolites is assigned to each metabolite. In Saccharomyces Genome Database (SGD), which is a genetic database of yeast, an ID (gene ID) for identifying genes is assigned to each gene. Therefore, when the analyte is a metabolite or a gene, the metabolite ID and the gene ID can be included in the analytical data together with their names. In SGD, a PMID (ID assigned to each literature by PubMed) related to a gene is assigned to the gene together with the gene ID. Since the PMID is associated with the MeSHID assigned to the literature to which the PMID is assigned (the ID assigned to the MeSH term that manages documents accumulated in MEDLINE), when the analyte is a yeast gene, the gene ID, PMID, and MeSHID can be included in the analytical data together with the name.

### [Processing in Analytical Device]

Next, processing in the analytical device 50 will be described using a case where MEDLINE is used as a literature database as an example.

### First Embodiment

FIG. 5 is a flowchart illustrating an example of processing of the analytical device 50.

The analytical data transmitted from the terminal device is input to the control unit 51 via the communication unit 54 of the analytical device 50. The control unit 51 refers to the analytical data to acquire identification information (corresponding to the first identification information of the present invention) for identifying the analyte (step 101). Therefore, in the present embodiment, the control unit 51 functions as information acquisition unit.

When MEDLINE is used as the literature database, the identification information acquired in step 101 is MeSHID. Therefore, in a case where MEDLINE is used and MeSHID included in the analytical data, the control unit 51 acquires MeSHID from the analytical data. On the other hand, when the analytical data does not include MeSHID, an ID conversion tool (not illustrated) for converting the name of the analyte and the ID (Metabolite ID, Gene ID, and the like) of the analyte into MeSHID is installed in advance in the analytical device 50. Then, the control unit 51 converts the name or ID of the analyte acquired from the analytical data into MeSHID using the ID conversion tool. Alternatively, under the control of the control unit 51, the inquiry unit 55 may make an inquiry to PubMed to acquire the MeSHID corresponding to the name or ID of the analyte acquired from the analytical data.

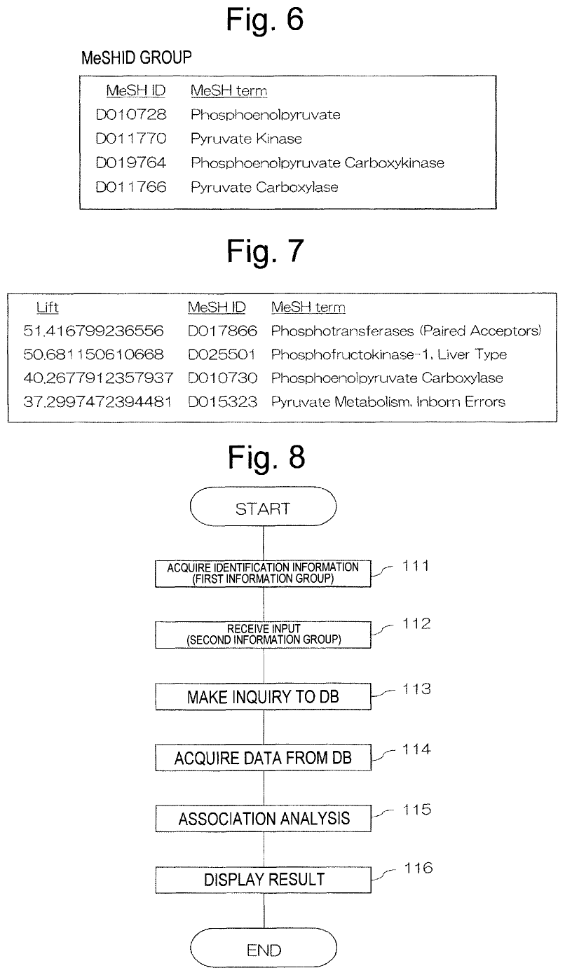

When the identification information (MeSHID) is acquired for all the analytes contained in the sample, the inquiry unit 55 subsequently makes an inquiry to PubMed (database) to acquire co-occurrence data related to the MeSHID of the analyte (step 102). Specifically, a set of MeSHID of all the objects to be analyzed (hereinafter, referred to as a MeSHID group; see FIG. 6) is output from the analysis device 50 via the communication unit 54 and transmitted to the PubMed side via the Internet. Upon receiving the MeSHID group, PubMed obtains co-occurrence data in literatures accumulated in MEDLINE of all MeSHIDs included in the MeSHID group from MEDLINE Co-Occurrence (MRCOC) (https://ii.nlm.nih.gov/MRCOC.shtml, [searched on Apr. 25, 2019]), which is one of services available via PubMed, and transmits the co-occurrence data to the analytical device 50. The co-occurrence data is composed of a text file (CoOccurs.txt) in which the MeSH term appearing simultaneously with all MeSH terms included in the MeSHID group, the MeSHID, and the value of the co-occurrence frequency are described in association with each other in the literatures accumulated in MEDLINE.

When the analytical device 50 acquires the co-occurrence data transmitted from PuBMed (step 103), the analysis unit 56 performs association analysis on the co-occurrence data (step 104). In association analysis, a related term of the analyte is extracted from co-occurrence data according to a rule adopting at least one of a confidence level, a support level, and a lift value. Therefore, in this embodiment, PubMed and the analysis unit 56 correspond to the extraction unit of the present invention.

Here, the related term means a term commonly related to all the analytes contained in the sample. Its specific examples include a term representing an attribute (kind, attribution, and the like) common to the analytes, a name of a certain metabolic pathway, a name of an enzyme, a gene, or the like involved in the metabolic pathway when the analyte is a metabolite of the metabolic pathway, a disease, a name of a causative substance other than the analyte when the analyte is a causative substance related to a specific disease, and the like. In the following description, it is assumed that MeSHID or MeSH term is extracted as a related term.

The display control unit 57 displays the result of the association analysis on the display unit 59 (step 105). Therefore, in the present embodiment, the display control unit 57 corresponds to presentation unit. FIG. 7 is an example of contents displayed on the display unit 59. In this example, as a result of association analysis according to the rule adopting the lift value, extracted related terms are displayed on the display unit 59. Specifically, a set of MeSHID and MeSH terms having a lift value of 30(%) or more is displayed side by side together with the lift value in descending order of the lift value. The lift value of each set is an average value of lift values calculated in combination with each of four MeSHIDs (see FIG. 6) included in the MeSHID group.

In the association analysis, a rule (recommendation rule) in which not only the lift value but also the confidence and the support levels are combined may be set, and the MeSHID to be extracted may be narrowed down. In addition, the lift value may not be displayed on the display unit 59 (that is, a set of MeSHID and MeSH term is displayed), and only the MeSH term or only the MeSHID may be displayed.

The user can search a literature database such as MEDLINE with reference to the list of MeSH terms and the like displayed on the display unit 59 to narrow down literatures meaningful for analysis of analysis data. For example, when only the MeSH term included in the MeSHID group illustrated in FIG. 6 is input in the keyword search of PubMed, in a case where a large number of literatures matching the search condition are extracted, the literatures can be narrowed down by adding an appropriate MeSH term to the keyword from the list of MeSH terms and the like displayed on the display unit 59.

It is easy to imagine that a literature describing metabolic pathways will be of reference, for example, when analyzing Saccharomyces cerevisiae metabolites. In this case, the MeSH term for narrowing is different depending on whether the user's interest is in a specific metabolite, a function of an enzyme involved in a metabolic reaction, a disease caused by metabolic disorders, or the like. On the other hand, in the present embodiment, a plurality of MeSH terms serving as keywords for searching the literature database and extracting the literature is extracted from the relevance with the analyte using the association analysis and presented to the user.

Therefore, for example, when the user has a strong interest in the mechanism of pyruvate metabolism, the MeSH term “Pyruvate Metabolism, Inborn Errors” related to pyruvate metabolism can be selected from the MeSH terms illustrated in FIG. 7, and the MeSH term can be added to the narrowed search of PubMed. In this way, it is possible to efficiently extract literatures related to pyruvate metabolism in congenital metabolic disorders.

### Second Embodiment

FIG. 8 is a flowchart illustrating another example of the processing of the analytical device 50.

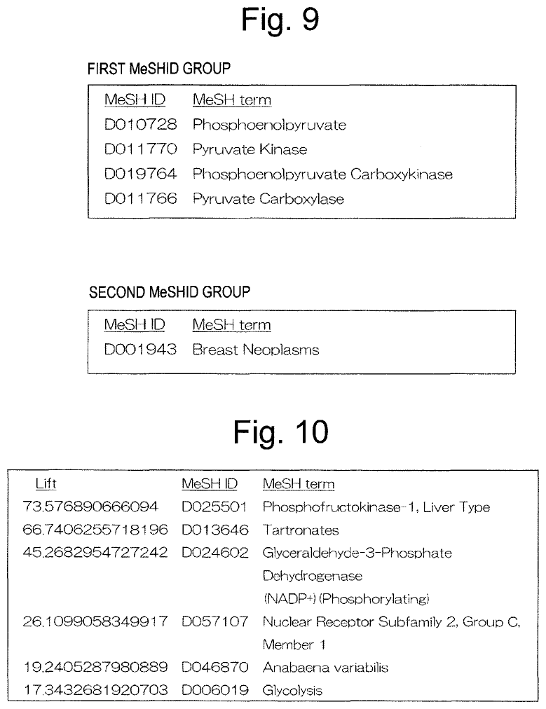

When the analytical device 50 receives the analytical data via the communication unit 54, the control unit 51 acquires identification information for identifying the analyte from the analytical data (step 111). Also in this embodiment, similarly to the first embodiment, it is assumed that the identification information acquired by the control unit 51 is MeSHID. FIG. 9 illustrates a set of MeSHIDs acquired in the present embodiment. The set of MeSHID shown in FIG. 9 is hereinafter referred to as a “first MeSHID group”.

Next, the control unit 51 receives an input of the second identification information from the user via the input unit 58 (step 112). The second identification information is a term appropriately selected by the user according to the purpose of measuring the analyte contained in the sample, the type of the sample, and the like, and its examples include terms such as diseases, biological species, organs, races, and the like. In the present embodiment, the “second MeSHID group” illustrated in FIG. 9 corresponds to second identification information. The character string input by the user may be either MeSHID or MeSH term. In the example shown in FIG. 9, it is assumed that “Breast Neoplasms” which is a MeSH term for breast cancer is input. Note that terms representing “cancer” include “Cancer”, “Tumor”, and “Neoplasma”, but in the thesaurus of MeSH, the notation is unified by assigning the MeSH term “Neoplasms” to papers dealing with cancer. Therefore, in a case where a term other than the MeSH term is input as the second identification information by the user, for example, the inquiry unit 55 may make an inquiry to PubMed to acquire the corresponding MeSH term or MeSHID. Furthermore, PubMed that has received an inquiry from the inquiry unit 55 in the next step may be converted into MeDHID.

When the control unit 51 acquires the first identification information and the second identification information, the inquiry unit 55 makes an inquiry to PubMed (database) to acquire a term related to MeSHID included in the first and second identification information (Step 113). Also in this embodiment, similarly to the first embodiment, PubMed transmits co-occurrence data obtained from MRCOC to the analysis unit 56.

When co-occurrence data is acquired from the database 41 (step 114), the analysis unit 56 performs association analysis on the co-occurrence data (step 115). Since contents of the association analysis are the same as those in the first embodiment, their description is omitted. In this embodiment, unlike the first embodiment, since the first MeSHID group and the second MeSHID group are transmitted to PubMed, co-occurrence data provided from PubMed is co-occurrence data common to the first MeSHID group and the second MeSHID group. Specifically, in literatures related to breast cancer that is the second identification information among the literatures recorded in MEDLINE, MeSH terms that appear simultaneously with MeSH terms included in the first MeSHID group are included in co-occurrence data.

The display control unit 57 displays the result of the association analysis on the display unit 59 (step 116). FIG. 10 is an example of contents displayed on the display unit 59. In this example, a set of MeSHID and MeSH terms having a lift value of 15(%) or more is displayed side by side together with the lift value in descending order of the lift value.

In this embodiment, since a request from the user to obtain breast cancer-related information is reflected in the co-occurrence data, as shown in FIG. 10, the information displayed on the display unit 59 as a result of association analysis includes information highly related to breast cancer. For example, the second MeSH term “Tartronates” from the top in the list shown in FIG. 10 is included in literatures reporting research results using human breast cancer-derived cell lines as inhibitors of pyruvate metabolism. Therefore, even a user who is not familiar with drugs can know the name of the inhibitor.

As described above, in the present embodiment, since the user can input the second identification information, information unnecessary for extracting the related term of the analyte can be excluded in advance.

### Modifications

In the above embodiment, the analytical device 50 includes one personal computer, but some of the functional blocks of the analytical device 50 may be mounted on a terminal device such as another personal computer or a tablet terminal connected to the analytical device 50 via a communication line. Furthermore, software that is an entity of each functional block of the analytical device 50 may be stored in an application server connected to the analytical device 50 via a communication line, and the software may be downloaded from the application server to the analytical device 50 as necessary.

As the input unit, not only the input unit 58 of the analytical device 50 but also an input device of a terminal device connected via the Internet 20 may be used. A computer that executes the analytical method described in the above embodiment proposes terms beyond a user's assumption by displaying keywords or IDs recommended for collecting information for analyzing analysis data. For this reason, the computer is also an information collection support device from another viewpoint.

In this embodiment, MRCOC provided on PubMed is used to acquire co-occurrence data, but the analytical device 50 may have a function of generating co-occurrence data. By generating co-occurrence data by adopting a co-occurrence index (for example, Dice coefficient, Jaccard coefficient, Simpson coefficient, Confidence, and the like) suitable for each database of document data, it is possible to improve the meaningfulness of related terms as search narrowing candidates.

In the above embodiment, association analysis is performed by the analysis unit 56, but the analysis method is not limited thereto. Association analysis is an analysis method suitable for related discovery among data mining methods for finding out a correlation or a pattern between pieces of data from enormous data. In this embodiment, association analysis is adopted because it is desired to find a term having a high correlation from terms used in a literature for a term queried in a database.

In the above embodiment, PubMed is used as the database of the document data, but for example, another database such as a literature information providing service operated by a publishing company or the like may be used. In this case, in the preprocessing, the content of the biological sample in the analysis data is specified by the keyword and the ID according to the thesaurus used to classify the literatures in the database. In addition, not only an existing database that can be used via the Internet but also a database that is independently constructed may be used via an arbitrary communication line.

In the above embodiment, the result of the association analysis is displayed on the display unit 59, but the result may be printed on paper or output by voice.

### [Aspects]

It is understood by those skilled in the art that the exemplary embodiments described above are specific examples of the following aspects.

(Item 1) An analytical device according to a first aspect of the present invention includes: an information acquisition unit configured to acquire first identification information for identifying, from a result of measuring an analyte contained in a sample using an analyzer, the analyte; an extraction unit configured to extract a related term related to the analyte, from a database in which document data is accumulated, on a basis of the first identification information acquired by the information acquisition unit; and a presentation unit configured to present the related term acquired by the extraction unit to a user.

(Item 8) An analytical method according to a second aspect of the present invention includes: a step of acquiring a result of measuring an analyte contained in a sample using an analyzer; a step of acquiring first identification information for identifying the analyte from the result of measuring the analyte; a step of extracting a related term related to the analyte from a database in which document data is accumulated on a basis of the first identification information; and a presentation step of presenting the related term to a user.

According to the analytical device according to item 1 and the analytical method according to item 8, the related term of the analyte is extracted from the database in which the document data is accumulated using the first identification information identifying the analyte acquired from the measurement result of the analyte contained in the sample, and the term is presented to the user. One or more related terms may be presented to the user. Using the first identification information and the related term, the user can easily find the document data meaningful for understanding the measurement result of the analyte from the database.

(Item 2) The analytical device according to item 1, wherein the information acquisition unit is configured to acquire first identification information corresponding to each of a plurality of analytes, and the extraction unit is configured to extract related terms commonly related to the plurality of analytes.

(Item 9) The analytical method according to item 8, wherein the step of acquiring the information is a step of acquiring first identification information corresponding to each of a plurality of analytes, and the extracting step is a step of extracting a related term commonly related to the plurality of pieces of first identification information.

According to the analytical device according to item 2 and the analytical method according to item 9, the user can easily find the document data commonly related to the plurality of analytes from the database. For example, the mass spectrometer can collectively measure a plurality of analytes contained in a sample at one time. In the analytical device according to item 2 and the analytical method according to item 9, document data meaningful for understanding the measurement result of an analyzer capable of simultaneously measuring a plurality of analytes, such as a mass spectrometer, can be presented to the user.

(Item 3) The analytical device according to item 1, further including a reception unit configured to receive an input from a user, wherein the information acquisition unit is configured to acquire second identification information received by the reception unit, and the extraction unit is configured to extract the related term on a basis of both the first identification information and the second identification information.

(Item 10) The analytical method according to item 8, further including: a reception step of receiving an input of second identification information from a user; and a step of acquiring the second identification information received in the reception step, wherein the extraction step is a step of extracting the related term on a basis of both the first identification information and the second identification information.

In the analytical device according to item 3 and the analytical method according to item 10, the second identification information means information necessary for searching the database for document data meaningful for understanding the measurement result of the analyte, and reflects the intention of the user such as the purpose of measuring the analyte and the research field. Therefore, in the analytical device according to item 3, it is possible to extract related terms of the analyte by narrowing the range in which the user is interested.

(Item 5) The analytical device according to item 1, wherein the extraction unit is configured to extract the related term using a data mining analysis method.

(Item 12) The analytical method according to item 8, wherein the extracting step is a step of extracting the related term using a data mining analysis method.

According to the analytical device according to item 5 and the analytical method according to item 12, using a data mining analysis method, it is possible to present, to the user, related terms for acquiring meaningful document data beyond the user's assumption.

(Item 6) In the analytical device according to item 5, the extraction unit is configured to extract the related term using association analysis.

(Item 13) The analytical method according to item 12, wherein the extracting step is a step of extracting the related term using association analysis.

(Item 7) In the analytical device according to item 6, the extraction unit is configured to extract the related term according to a rule that adopts at least one of a confidence level, a support level, and a lift value in the association analysis.

(Item 14) In the analytical method according to item 13, the extracting step is a step of extracting the related term according to a rule that adopts at least one of a confidence level, a support level, and a lift value in the association analysis.

(Item 15) A program for causing a computer to execute: processing of acquiring a result of measuring an analyte contained in a sample using an analyzer; processing of acquiring first identification information for identifying the analyte, from a measurement result of the analyte; processing of extracting a related term related to the analyte, from a database in which document data is accumulated on a basis of the first identification information; and processing of presenting the related term to a user.

(Item 16) A computer-readable (non-transitory) storage medium recording a program for causing a computer to execute: processing of acquiring a result of measuring an analyte contained in a sample using an analyzer; processing of acquiring first identification information for identifying the analyte, from a measurement result of the analyte; processing of extracting a related term related to the analyte, from a database in which document data is accumulated on a basis of the first identification information; and processing of presenting the related term to a user.

Note that, the above description is for explaining the embodiment of the present invention, and is not for limiting the scope of the present invention.

### REFERENCE SIGNS LIST

10 . . . Analyzer
11 . . . Device Main Body
12 . . . Personal Computer
20 . . . Internet
21 . . . Tablet Terminal
22 . . . Personal Computer
31 . . . Statistical Tool
32 . . . Mapping Tool
33 . . . Node Extraction Tool
41 . . . Database
42 . . . Database
43 . . . Database
44 . . . Database
50 . . . Analytical Device
51 . . . Control Unit
52 . . . Arithmetic Device
53 . . . Auxiliary Storage Device
54 . . . Communication Unit
55 . . . Inquiry Unit
56 . . . Analysis Unit
57 . . . Display Control Unit
58 . . . Input Unit
59 . . . Display Unit
60 . . . Device Main Body

1. An analytical device comprising:
an information acquisition unit configured to acquire first identification information, for identifying, from a result of measuring an analyte contained in a sample using an analyzer, the analyte;
an extraction unit configured to extract a related term related to the analyte, from a database in which document data is accumulated, on a basis of the first identification information acquired by the information acquisition unit; and
a presentation unit configured to present the related term acquired by the extraction unit to a user.

2. The analytical device according to claim 1, wherein
the information acquisition unit is configured to acquire first identification information corresponding to each of a plurality of analytes, and
the extraction unit is configured to extract a related term commonly related to the plurality of analytes.

3. The analytical device according to claim 1, further comprising
a reception unit configured to receive an input from a user, wherein
the information acquisition unit is configured to acquire second identification information received by the reception unit, and
the extraction unit is configured to extract the related term on a basis of both the first identification information and the second identification information.

4. The analytical device according to claim 1, wherein
the analyzer is a mass spectrometer.

5. The analytical device according to claim 1, wherein
the extraction unit is configured to extract the related term using a data mining analysis method.

6. The analytical device according to claim 5, wherein
the extraction unit is configured to extract the related term using association analysis.

7. The analytical device according to claim 6, wherein
the extraction unit is configured to extract the related term according to a rule that adopts at least one of a confidence level, a support level, and a lift value in the association analysis.

8. An analytical method comprising:
a step of acquiring a result of measuring an analyte contained in a sample using an analyzer;
a step of acquiring first identification information for identifying the analyte from the result of measuring the analyte;
a step of extracting a related term related to the analyte from a database in which document data is accumulated on a basis of the first identification information; and
a presentation step of presenting the related term to a user.

9. The analytical method according to claim 8, wherein
the step of acquiring the information is a step of acquiring first identification information corresponding to each of a plurality of analytes, and
the extracting step is a step of extracting a related term commonly related to the plurality of pieces of first identification information.

10. The analytical method according to claim 8, further comprising:
a reception step of receiving an input of second identification information from a user; and
a step of acquiring second identification information received in the reception step, wherein
the extracting step includes extracting the related term on a basis of both the first identification information and the second identification information.

11. The analytical method according to claim 8, wherein
the analyzer is a mass spectrometer.

12. The analytical method according to claim 8, wherein
the extracting step is a step of extracting the related term using a data mining analysis method.

13. The analytical method according to claim 12, wherein
the extracting step is a step of extracting the related term using association analysis.

14. The analytical method according to claim 13, wherein
the extracting step is a step of extracting the related term according to a rule that adopts at least one of a confidence level, a support level, and a lift value in the association analysis.
